(19) United States
(12) Patent Application Publication
(10) Pub. No.: US 2026/0125417 A1
(43) Pub. Date: May 7, 2026

(54) GLUCOCORTICOID RECEPTOR AGONISTS

(71) Applicant: ELI LILLY AND COMPANY, Indianapolis, IN (US)

(72) Inventors: Adel Ahmed Rashad AHMED, Fishers, IN (US); Joshua Ryan CLAYTON, Fishers, IN (US); Jose Eduardo LOPEZ GARCIA, Fishers, IN (US); Jothirajah MARIMUTHU, Carmel, IN (US); William Thomas MCMILLEN, McCordsville, IN (US); Ryan Edward STITES, Indianapolis, IN (US); Takako WILSON, San Diego, CA (US); Jacqueline Mary WURST, Zionsville, IN (US)

(21) Appl. No.: 19/114,373

(22) PCT Filed: Sep. 21, 2023
(86) PCT No.: PCT/US2023/074725

§ 371 Date: Mar. 21, 2025

Related U.S. Application Data
(60) Provisional application No. 63/409,024, filed on Sep. 22, 2022.

(51) Int. Cl. C07J 71/00; A61K 31/58; C07B 59/00

(52) U.S. Cl. CPC C07J 71/0031; A61K 31/58; C07B 59/007; C07B 2200/05

(57) ABSTRACT

The present invention provides a compound of Formula I:

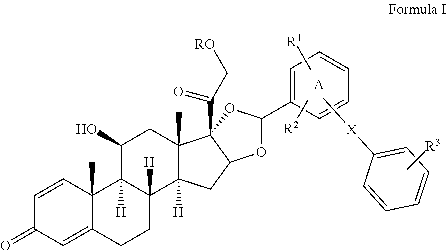

wherein R is H or

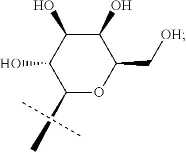

R¹ is H, halogen, C1-C3 alkyl, C3-C6 cycloalkyl, C1-C3 alkoxy, C2-C3 alkenyl, OCF₃,

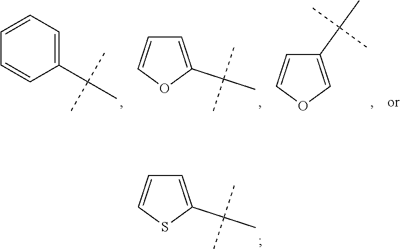

R² is H, halogen, C1-C3 alkyl, C1-C3 alkoxy, or C2-C4 alkenyl;
R³ is NH₂, or CH₂NH₂; and
X is O, OCH₂, OCH₂CH₂, OCH(CH₃), CH₂O, SCH₂, CH₂S, CH₂, NHCH₂, CH₂NH, N(CH₃)CH₂, CH₂CH₂, C≡C, or a bond, wherein X is connected to phenyl ring A at the ortho or the meta position, or a pharmaceutically acceptable salt thereof, wherein the compound of Formula I, or pharmaceutically acceptable salt thereof is useful for treating autoimmune and inflammatory diseases, such as atopic dermatitis and rheumatoid arthritis.

The present disclosure provides compounds that are glucocorticoid receptor agonists and are useful for the treatment of autoimmune and inflammatory diseases, such as atopic dermatitis, inflammatory bowel disease, systemic lupus erythematosus, lupus nephritis, and rheumatoid arthritis, processes for preparing these compounds, pharmaceutical compositions comprising these compounds, and methods of using these compounds and compositions are also provided.

Atopic dermatitis is a chronic, pruritic relapsing and remitting inflammatory skin disease that occurs frequently in children, but also affects many adults. Current treatments of atopic dermatitis include light therapy, topical creams containing corticosteroids or calcineurin inhibitors, or a subcutaneous injectable biologic known as dupilumab. In spite of progress made in treating atopic dermatitis, there remains a significant need for new compounds to treat atopic dermatitis and other inflammatory and autoimmune diseases.

WO2017/210471 discloses certain glucocorticoid receptor agonists and immunoconjugates thereof useful for treating autoimmune or inflammatory diseases. WO2018/089373 discloses novel steroids, protein conjugates thereof, and methods for treating diseases, disorders, and conditions comprising administering the steroids and conjugates.

The present invention provides certain novel compounds which are glucocorticoid receptor agonists. The present invention further provides certain novel compounds which are prodrugs of glucocorticoid receptor agonists. In addition, the present invention provides certain novel compounds which are glucocorticoid receptor agonists useful in the treatment of autoimmune and inflammatory diseases such as atopic dermatitis, inflammatory bowel disease, rheumatoid arthritis, systemic lupus erythematosus, and lupus nephritis.

Accordingly, in one embodiment, the invention provides a compound of Formula I:

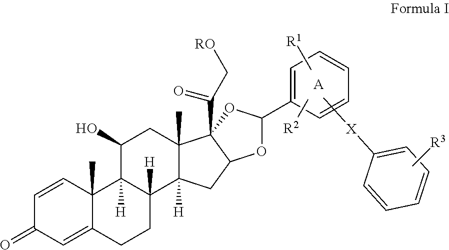

wherein R is H or

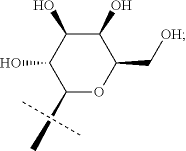

R¹ is H, halogen, CN, C1-C3 alkyl, C3-C6 cycloalkyl, C1-C3 alkoxy, C2-C3 alkenyl, OCF₃,

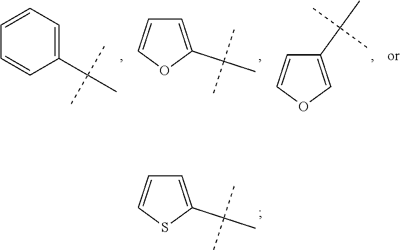

R² is H, halogen, C1-C3 alkyl, C1-C3 alkoxy, or C2-C3 alkenyl;
R³ is NH₂, or CH₂NH₂; and
X is O, OCH₂, OCH₂CH₂, CH₂O, SCH₂, CH₂S, CH₂, NHCH₂, CH₂NH, N(CH₃)CH₂, CH₂CH₂, C≡C, or a bond, wherein X is connected to phenyl ring A at the ortho or the meta position, or a pharmaceutically acceptable salt thereof.

In one embodiment, the invention provides a compound of Formula Ia:

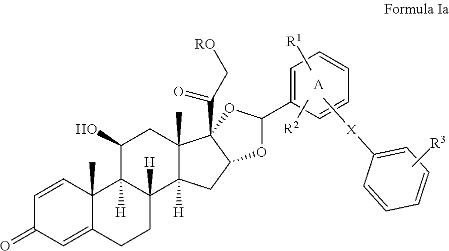

wherein R is H or

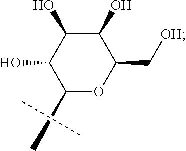

R¹ is H, halogen, C1-C3 alkyl, C3-C6 cycloalkyl, C1-C3 alkoxy, C2-C3 alkenyl, OCF₃,

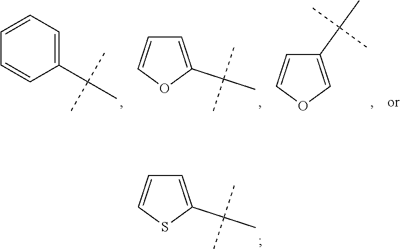

R² is H, halogen, C1-C3 alkyl, C1-C3 alkoxy, or C2-C4 alkenyl;
R³ is NH₂, or CH₂NH₂; and
X is O, OCH₂, OCH₂CH₂, OCH₂C≡C, OCH(CH₃), CH₂O, SCH₂, CH₂S, CH₂, NHCH₂, CH₂NH, N(CH₃)CH₂, CH₂CH₂, C≡C, or a bond, wherein X is connected to phenyl ring A at the ortho or the meta position, or a pharmaceutically acceptable salt thereof.

In one embodiment, the invention provides a compound of Formula Ib:

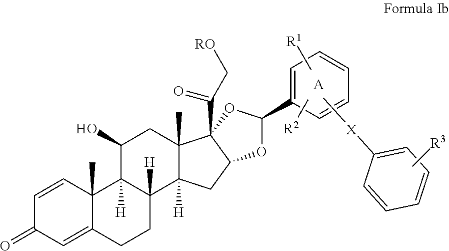

wherein R is H or

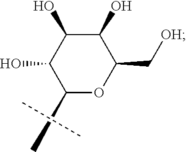

R¹ is H, halogen, C1-C3 alkyl, C3-C6 cycloalkyl, C1-C3 alkoxy, C2-C3 alkenyl, OCF₃,

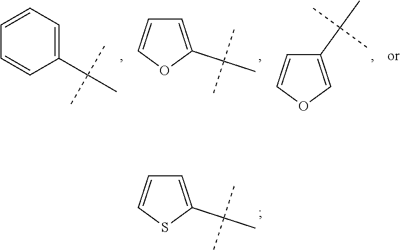

R² is H, halogen, C1-C3 alkyl, C1-C3 alkoxy, or C2-C4 alkenyl;
R³ is NH₂, or CH₂NH₂; and
X is O, OCH₂, OCH₂CH₂, OCH(CH₃), CH₂O, SCH₂, CH₂S, CH₂, NHCH₂, CH₂NH, N(CH₃)CH₂, CH₂CH₂, C≡C, or a bond, wherein X is connected to phenyl ring A at the ortho or the meta position, or a pharmaceutically acceptable salt thereof.

In one embodiment, the invention provides a compound of Formula Ic:

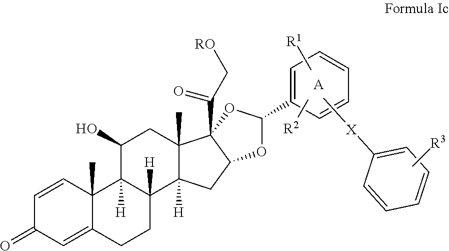

wherein R is H or

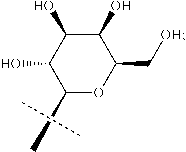

R¹ is H, halogen, C1-C3 alkyl, C3-C6 cycloalkyl, C1-C3 alkoxy, C2-C3 alkenyl, OCF₃,

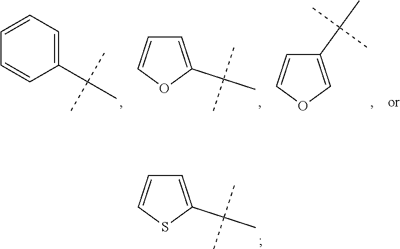

R² is H, halogen, C1-C3 alkyl, C1-C3 alkoxy, or C2-C4 alkenyl;
R³ is NH₂, or CH₂NH₂; and
X is O, OCH₂, OCH₂CH₂, OCH(CH₃), CH₂O, SCH₂, CH₂S, CH₂, NHCH₂, CH₂NH, N(CH₃)CH₂, CH₂CH₂, C≡C, or a bond, wherein X is connected to phenyl ring A at the ortho or the meta position, or a pharmaceutically acceptable salt thereof.

In an embodiment, the invention provides a compound of Formula Ib(i):

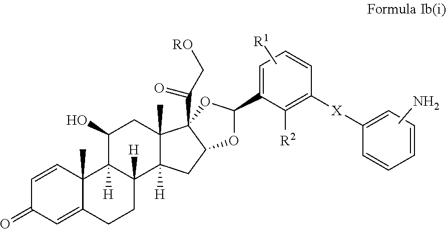

or a pharmaceutically acceptable salt thereof.

In an embodiment, the invention provides a compound of Formula Ic(i):

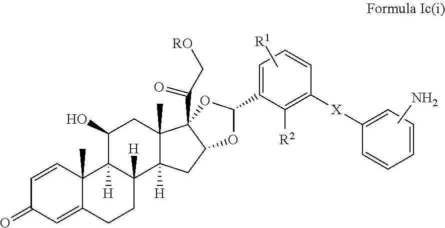

or a pharmaceutically acceptable salt thereof.

In an embodiment, the invention provides a compound of Formula Ib(ii):

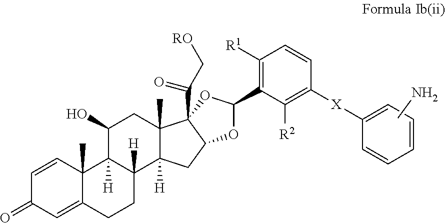

or a pharmaceutically acceptable salt thereof.

In an embodiment, the invention provides a compound of Formula Ic(ii):

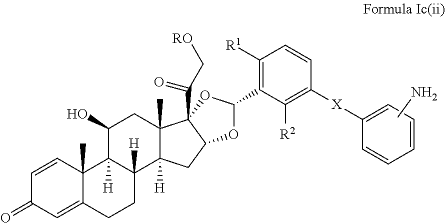

or a pharmaceutically acceptable salt thereof.

In an embodiment, the invention provides a compound of Formula Ib(iii):

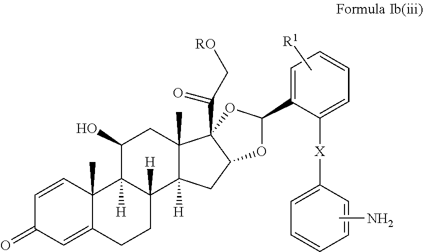

or a pharmaceutically acceptable salt thereof.

In an embodiment, the invention provides a compound of Formula Ic(iii):

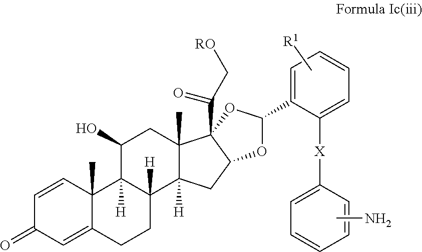

or a pharmaceutically acceptable salt thereof.

In one embodiment, the invention provides a compound of Formula II:

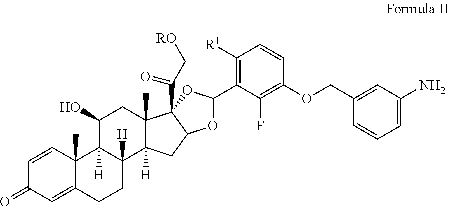

wherein R is H or

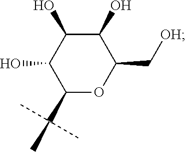

and
R¹ is —CH₃ or —OCH₃,
or a pharmaceutically acceptable salt thereof.

In a particular embodiment, the invention provides a compound of Formula IIa:

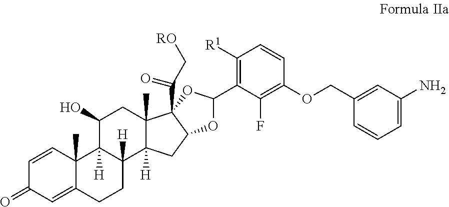

wherein R is H or

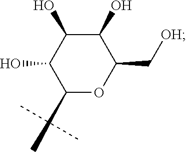

and
R¹ is —CH₃ or —OCH₃,
or a pharmaceutically acceptable salt thereof.

In a particular embodiment, the invention provides a compound of Formula IIb:

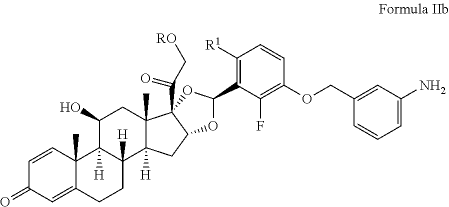

wherein R is H or

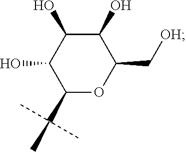

and
R¹ is —CH₃ or —OCH₃, or a pharmaceutically acceptable salt thereof.

In a particular embodiment, the invention provides a compound of Formula IIc:

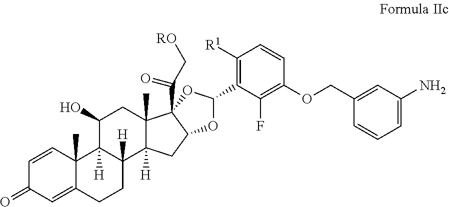

wherein R is H or

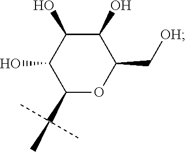

and
R¹ is —CH₃ or —OCH₃, or a pharmaceutically acceptable salt thereof.

In an embodiment, the invention provides a compound of Formula III:

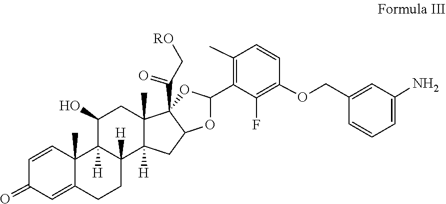

wherein R is H or

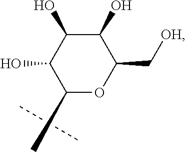

or a pharmaceutically acceptable salt thereof.

In a particular embodiment, the invention provides a compound of Formula IIIa:

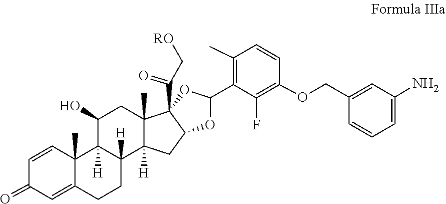

wherein R is H or

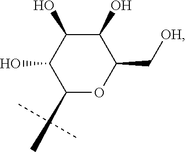

or a pharmaceutically acceptable salt thereof.

In a particular embodiment, the invention provides a compound of Formula IIIb:

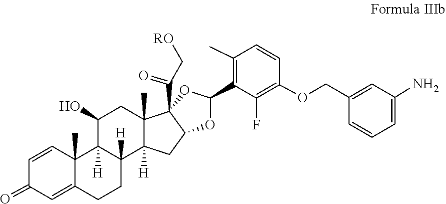

wherein R is H or

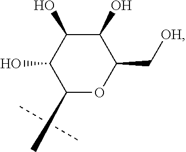

or a pharmaceutically acceptable salt thereof.

In a particular embodiment, the invention provides a compound of Formula IIIc:

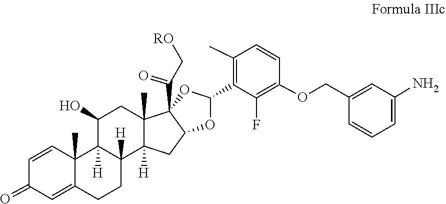

wherein R is H or

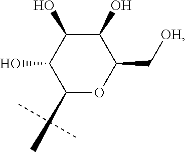

or a pharmaceutically acceptable salt thereof.

In an embodiment, R is H.

In an embodiment, R¹ is F, CH₂CH₃, OCH₃, or OC(²H)₃. In an embodiment, R¹ is F In an embodiment, R¹ is CH₂CH₃. In an embodiment, R¹ is OCH₃. In an embodiment, R¹ is OC(²H)₃.

In an embodiment, R² is F, CH₂CH₃, OCH₃, or OC(²H)₃. In an embodiment, R² is F In an embodiment, R² is CH₂CH₃. In an embodiment, R² is OCH₃. In an embodiment, R² is OC(²H)₃.

In an embodiment, X is CH₂CH₂, OCH₂, or OCH₂CH₂. In an embodiment, X is CH₂CH₂. In an embodiment, X is OCH₂. In an embodiment, X is OCH₂CH₂.

In an embodiment, R³ is NH₂.

In an embodiment, X is connected to phenyl ring A at the meta position.

In an embodiment, X is connected to phenyl ring A at the ortho position.

In an embodiment, the present invention also provides a method of treating an inflammatory disease in a patient in need of such treatment, comprising administering to the patient an effective amount of a compound of Formula I, or a pharmaceutically acceptable salt thereof. In an embodiment, the present invention also provides a method of treating atopic dermatitis in a patient in need of such treatment, comprising administering to the patient an effective amount of a compound of Formula I, or a pharmaceutically acceptable salt thereof. In an embodiment, the present invention further provides a method of treating inflammatory bowel disease in a patient in need of such treatment, comprising administering to the patient an effective amount of a compound of Formula I, or a pharmaceutically acceptable salt thereof. In an embodiment, the present invention further provides a method of treating rheumatoid arthritis in a patient in need of such treatment, comprising administering to the patient an effective amount of a compound of Formula I, or a pharmaceutically acceptable salt thereof. In an embodiment, the present invention also provides a method of treating systemic lupus erythematosus in a patient in need of such treatment, comprising administering to the patient an effective amount of a compound of Formula I, or a pharmaceutically acceptable salt thereof. In an embodiment, the present invention also provides a method of treating lupus nephritis in a patient in need of such treatment, comprising administering to the patient an effective amount of a compound of Formula I, or a pharmaceutically acceptable salt thereof.

In an embodiment, the present invention further provides a compound of Formula I, or a pharmaceutically acceptable salt thereof for use in therapy. In an embodiment, the present invention provides a compound of Formula I, or a pharmaceutically acceptable salt thereof for use in treating an inflammatory disease. In an embodiment, the present invention provides a compound of Formula I, or a pharmaceutically acceptable salt thereof, for use in treating atopic dermatitis. In an embodiment, the present invention provides a compound of Formula I, or a pharmaceutically acceptable salt thereof, for use in treating rheumatoid arthritis. In an embodiment, the present invention provides a compound of Formula I, or a pharmaceutically acceptable salt thereof, for use in treating inflammatory bowel disease. In an embodiment, the present invention provides a compound of Formula I, or a pharmaceutically acceptable salt thereof, for use in treating lupus nephritis. In an embodiment, the present invention provides a compound of Formula I, or a pharmaceutically acceptable salt thereof, for use in treating systemic lupus erythematosus.

In an embodiment, the present invention also provides the use of a compound of Formula I, or a pharmaceutically acceptable salt thereof, for the manufacture of a medicament for treating an inflammatory disease. In an embodiment, the present invention provides the use of a compound of Formula I, or a pharmaceutically acceptable salt thereof, for the manufacture of a medicament for treating atopic dermatitis. In an embodiment, the present invention provides the use of a compound of Formula I, or a pharmaceutically acceptable salt thereof, for the manufacture of a medicament for treating rheumatoid arthritis. In an embodiment, the present invention further provides the use of a compound of Formula I, or a pharmaceutically acceptable salt thereof, for the manufacture of a medicament for treating inflammatory bowel disease. In an embodiment, the present invention further provides the use of a compound of Formula I, or a pharmaceutically acceptable salt thereof, for the manufacture of a medicament for treating lupus nephritis. In an embodiment, the present invention also provides the use of a compound of Formula I, or a pharmaceutically acceptable salt thereof, for the manufacture of a medicament for treating systemic lupus erythematosus.

In an embodiment, the present invention further provides a pharmaceutical composition, comprising a compound of Formula I, or a pharmaceutically acceptable salt thereof, with one or more pharmaceutically acceptable carriers, diluents, or excipients. In an embodiment, the present invention further provides a process for preparing a pharmaceutical composition, comprising admixing a compound of Formula I, or a pharmaceutically acceptable salt thereof, with one or more pharmaceutically acceptable carriers, diluents, or excipients. In an embodiment, the present invention also encompasses novel intermediates and processes for the synthesis of compounds of Formula I.

As used herein, the terms “treating”, “treatment”, or “to treat” includes restraining, slowing, stopping, or reversing the progression or severity of an existing symptom or disorder.

As used herein, the term “patient” refers to a mammal, in particular a human.

As used herein, the term “effective amount” refers to the amount or dose of compound of the invention, or a pharmaceutically acceptable salt thereof which, upon single or multiple dose administration to the patient, provides the desired effect in the patient under diagnosis or treatment.

An effective amount can be determined by one skilled in the art by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

As used herein, it is understood that Formula I encompasses Formulas Ia, Ib, Ic, Ib(i), Ic(i), Ib(ii), Ic(ii), Ib(iii), Ic(iii), II, Ia, IIb, IIc, III, IIa, IIIb, and IIIc, and all references to Formula I herein should be read as including Formulas Ia, Ib, Ic, Ib(i), Ic(i), Ib(ii), Ic(ii), Ib(iii), Ic(iii), II, IIa, IIb, IIc, III, IIIa, IIIb, and IIIc.

As used herein, it is understood that Formula II encompasses Formulas IIa, IIb, and IIc, and all references to Formula II herein should be read as including Formulas IIa, IIb, and IIc.

As used herein, it is understood that Formula III encompasses Formulas IIIa, IIIb, and IIIc, and all references to Formula III herein should be read as including Formulas IIIa, IIIb, and IIIc.

As used herein “halogen” refers to F, Cl, Br, and I.

As used herein “C1-C3 alkyl” refers to CH₃, CH₂CH₃, CH₂CH₂CH₃, and CH(CH₃)₂.

As used herein “C3-C6 cycloalkyl” refers to cyclopropyl, cyclobutyl, cyclopentyl, and cyclohexyl.

As used herein “C1-C3 alkoxy” refers to OCH₃, OCH₂CH₃, OCH₂CH₂CH₃, and OCH(CH₃)₂.

As used herein “C2-C3 alkenyl” refers to HC═CH₂, and C(CH₃)═CH₂.

As used herein, the ortho and meta positions on phenyl ring A are shown in Formula I below:

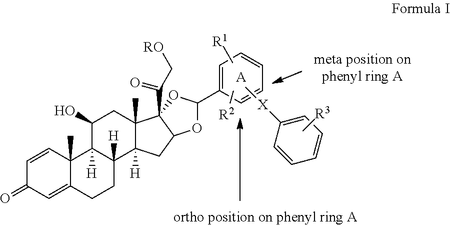

For example, the compound of Formula I′ illustrates X connected to phenyl ring A at the meta position:

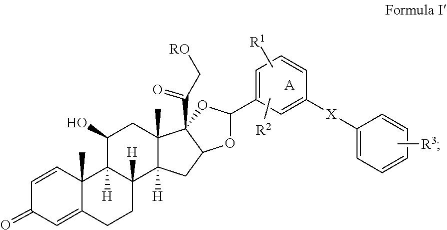

and the compound of Formula I″ illustrates X connected to phenyl ring A at the ortho position:

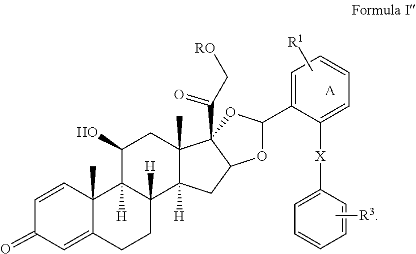

It is appreciated by one of ordinary skill in the art that when X is connected to phenyl ring A at the ortho position as shown in Formula I″, then R² is H.

In some embodiments, the compound is selected from the compounds described in Table I and pharmaceutically acceptable salts and stereoisomers thereof.

In some embodiments, the compound is selected from the compounds described in Table I and pharmaceutically acceptable salts thereof.

In some embodiments, the compound is selected from the compounds described in Table I.

TABLE I

| Compound | |
|---|---|
| No. | Structure |
| 1 | 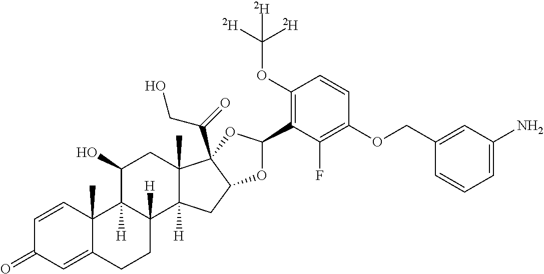 |
| | (6aR,6bS,7S,8aS,8bS,10S,11aR,12aS,12bS)-10-(3-((3- |
| | aminobenzyl)oxy)-2-fluoro-6-(methoxy-d3)phenyl)-7- |
| | hydroxy-8b-(2-hydroxyacetyl)-6a,8a-dimethyl- |
| | 1,2,6a,6b,7,8,8a,8b,11a,12,12a,12b-dodecahydro-4H- |
| | naphtho[2′,1′:4,5]indeno[1,2-d]\|1,3]dioxol-4-one |
| 2 | 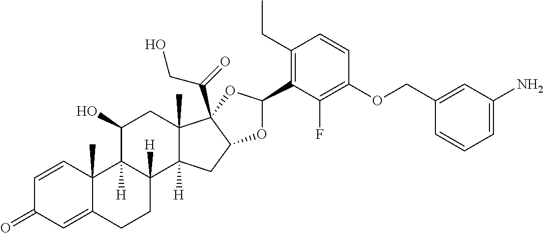 |
| | (6aR,6bS,7S,8aS,8bS,10S,11aR,12aS,12bS)-10-(3-((3- |
| | aminobenzyl)oxy)-6-ethyl-2-fluorophenyl)-7-hydroxy-8b- |
| | (2-hydroxyacetyl)-6a,8a-dimethyl- |
| | 1,2,6a,6b,7,8,8a,8b,11a,12,12a,12b-dodecahydro-4H- |
| | naphtho[2′,1′:4,5]indeno[1,2-d][1,3]dioxol-4-one |
| 3 | 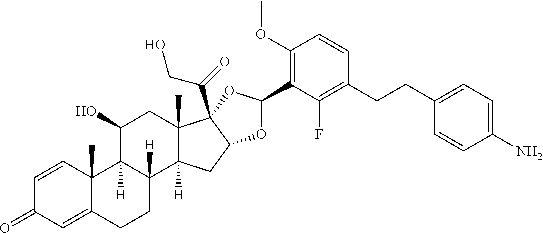 |
| | (6aR,6bS,7S,8aS,8bS,10S,11aR,12aS,12bS)-10-(3-(4- |
| | aminophenethyl)-2-fluoro-6-methoxyphenyl)-7-hydroxy- |
| | 8b-(2-hydroxyacetyl)-6a,8a-dimethyl- |
| | 1,2,6a,6b,7,8,8a,8b,11a,12,12a,12b-dodecahydro-4H- |
| | naphtho[2′,1′:4,5]indeno[1,2-d][1,3]dioxol-4-one |
| 4 | 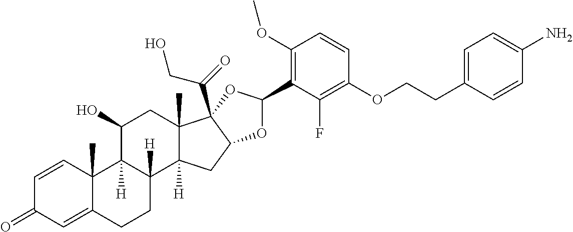 |
| | (6aR,6bS,7S,8aS,8bS,10S,11aR,12aS,12bS)-10-(3-(4- |
| | aminophenethoxy)-2-fluoro-6-methoxyphenyl)-7-hydroxy- |
| | 8b-(2-hydroxyacetyl)-6a,8a-dimethyl- |
| | 1,2,6a,6b,7,8,8a,8b,11a,12,12a,12b-dodecahydro-4H- |
| | naphtho[2′,1′:4,5]indeno[1,2-d][1,3]dioxol-4-one |
| 5 | 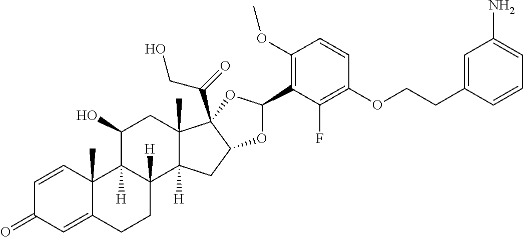 |
| | (6aR,6bS,7S,8aS,8bS,10S,11aR,12aS,12bS)-10-(3-(3- |
| | aminophenethoxy)-2-fluoro-6-methoxyphenyl)-7-hydroxy- |
| | 8b-(2-hydroxyacetyl)-6a,8a-dimethyl- |
| | 1,2,6a,6b,7,8,8a,8b,11a,12,12a,12b-dodecahydro-4H- |
| | naphtho[2′,1′:4,5]indeno[1,2-d][1,3]dioxol-4-one |

In some embodiments, the present disclosure provides a compound being an isotopic derivative (e.g., isotopically labeled compound) of any one of the compounds disclosed herein.

In some embodiments, the compound is an isotopic derivative of any one of the compounds described in Table I and pharmaceutically acceptable salts thereof.

In some embodiments, the compound is an isotopic derivative of any one of the compounds described in Table I.

It is understood that the isotopic derivative can be prepared using any of a variety of art-recognized techniques. For example, the isotopic derivative can generally be prepared by carrying out the procedures disclosed in the schemes and/or in the examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

In some embodiments, the compound is a deuterium labeled compound of any one of the compounds described in Table I and pharmaceutically acceptable salts thereof.

In some embodiments, the compound is a deuterium labeled compound of any one of the compounds described in Table I.

In addition, a compound of the present invention can be conjugated with an antibody to form an antibody drug conjugate (ADC) by methods understood by one of skill in the art. One example of such conjugation would include connection of a compound of the present invention to an antibody via a linker compound. Linker compounds known to those of skill in the art include, for example, cleavable linkers and noncleavable linkers. Such an ADC can deliver the compound of the present invention to specific target tissues or cells. Accordingly, provided herein are also ADCs comprising a compound of Formula I. In some embodiments, the compound of Formula I is conjugated to an antibody via a linker, e.g., a cleavable linker or a noncleavable linker.

The compounds or conjugates of the present invention can be formulated as pharmaceutical compositions administered by any route which makes the compound or conjugate bioavailable including, for example, oral, topical, or subcutaneous administration. Such pharmaceutical compositions, including ADCs, can be prepared using techniques and methods known in the art. Such pharmaceutical compositions, including ADCs can be prepared using techniques and methods known in the art (See, e.g., Remington: The Science and Practice of Pharmacy, A. Adejare, Editor, 23^{rd} Edition, published 2020, Elsevier Science; WO 2017/062271, and WO 2017/210471).

Furthermore, compounds of the present invention that have the hydroxy group at C21 capped wherein R is:

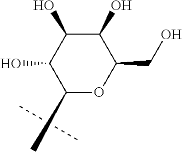

behave as prodrugs and are metabolized in vitro or in vivo to provide the active glucocorticoid receptor agonist wherein R is H.

Included within the scope of the present invention is a pharmaceutically acceptable salt of Formula I. A pharmaceutically acceptable salt of a compound of the invention, such as a compound of Formula I can be formed, for example, by reaction of an appropriate free base of a compound of the invention with an appropriate pharmaceutically acceptable acid in a suitable solvent such as diethyl ether under standard conditions well known in the art. See, for example, Berge, S. M., et al., “Pharmaceutical Salts,” Journal of Pharmaceutical Sciences, 66: 1-19, (1977).

Certain compounds described in the following preparations may contain a suitable nitrogen protecting group referred to herein as “Pg”. It is understood that protecting groups may be varied as appreciated by one of skill in the art depending on the particular reaction conditions and the particular transformations to be performed. The protection and deprotection conditions are well known to the skilled artisan and are described in the literature (See for example “Greene's Protective Groups in Organic Synthesis”, Fourth Edition, by Peter G. M. Wuts and Theodora W. Greene, John Wiley and Sons, Inc. 2007).

### EXAMPLES

The compounds of the present invention, or salts thereof, may be readily prepared by a variety of procedures known to one of ordinary skill in the art, some of which are illustrated in the preparations and examples below. One of ordinary skill in the art recognizes that the specific synthetic steps for each of the routes described may be combined in different ways, or in conjunction with steps from different schemes, to prepare compounds of the invention, or salts thereof. The product of each step can be recovered by conventional methods well known in the art, including extraction, evaporation, precipitation, chromatography, filtration, trituration, and crystallization. All substituents unless otherwise indicated, are as previously defined. The reagents and starting materials are readily available to one of ordinary skill in the art. The following preparations, examples, and assays further illustrate the invention, but should not be construed to limit the scope of the invention in any way.

TABLE 1
Abbreviations and definitions

| Term | Definition |
|---|---|
| ACN | acetonitrile |
| aq | aqueous |
| Å | angstrom(s) |
| BOC/Boc | tert-butyloxycarbonyl |
| C18 | octadecylsilane |
| DCM | dichloromethane |
| DEA | diethylamine |
| DIBAL-H | diisobutylaluminum hydride |
| DMEA | dimethylethylamine |
| DMF | N,N-dimethylformamide |
| DMSO | dimethyl sulfoxide |
| dppf | 1,1′-bis(diphenylphosphino)ferrocene |
| equiv | equivalent(s) |
| ES/MS | electrospray mass spectrometry |
| EtOAc | ethyl acetate |
| EtOH | ethanol |
| FA | formic acid |
| g | gram(s) |
| h | hour(s) |
| HPLC | high performance liquid chromatography |
| IPA | isopropanol |
| IPAm | isopropylamine |
| L | liter(s) |
| LC | liquid chromatography |
| LCMS | liquid chromatography mass spectrometry |
| LDA | lithium diisopropylamide |
| M | molar |
| mbar | millibar(s) |
| MeOH | methanol |
| min | minute(s) |
| mL | milliliter(s) |
| mM | millimolar |
| mmol | millimole(s) |
| mol | mole(s) |
| MS | mass spectrometry |
| MTBE | methyl tert-butyl ether |
| MW | molecular weight |
| m/z | mass-to-charge ratio |
| nm | nanometer(s) |
| NMR | nuclear magnetic resonance |
| Pet ether | petroleum ether |
| ppm | parts per million |
| ROE | rotating-frame Overhauser enhancement |
| RP-HPLC | reverse-phase HPLC |
| rt | room temperature |
| satd | saturated |
| SFC | supercritical fluid chromatography |
| SM | starting material |
| THF | tetrahydrofuran |
| wt | weight |

### Example 1. Preparations of Pre-Cursors

### Preparation 1. Synthesis of tert-butyl (3-((2-fluoro-3-formyl-4-(methoxy-d₃)phenoxy)methyl)phenyl)carbamate

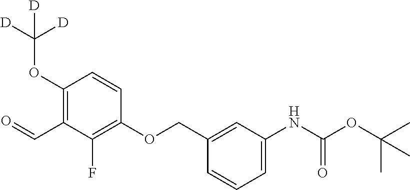 [SMILES: [2H]C([2H])([2H])Oc1ccc(OCc2cccc(NC(=O)OC(C)(C)C)c2)c(F)c1C=O]

A solution of 2-fluoro-3-hydroxy-6-(trideuteriomethoxy)benzaldehyde (870 mg, 5.0 mmol) in DMF (15 mL) was treated with potassium carbonate (2.2 g, 16 mmol) and stirred at rt for 15 min. Tert-butyl N-[3-(bromomethyl)phenyl]carbamate (1.5 g, 5.2 mmol) was added in one portion and the reaction was stirred at rt. After 18 h, the reaction mixture was partitioned between EtOAc and H₂O. The phases were separated, the organic phase was transferred to a flask, and the solvent was evaporated under vacuum. Residual DMF was evaporated with the help of xylenes. The crude residue was purified by normal phase purification, eluting with 0-50% EtOAc in hexanes to give the title compound (1.9 g, 90% yield). MS m/z 377.2 (M−H).

### Preparation 2. Synthesis of 2-fluoro-3-hydroxy-6-(methoxy-d₃)benzaldehyde

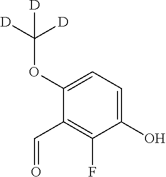 [SMILES: [2H]C([2H])([2H])Oc1ccc(O)c(F)c1C=O]

To a solution of 3-[tert-butyl(diphenyl)silyl]oxy-2-fluoro-6-(trideuteriomethoxy)benzaldehyde (7.3 g, 18 mmol) in THF (60 mL) was added TBAF (20 mL, 20 mmol, 1 mol/L in THF). The mixture was stirred at rt. After 18 h, the solvent was evaporated to give the crude product. The crude residue was purified by normal phase purification, eluting with 0-2% MeOH in DCM to give the title compound (3.1 g, 85% yield). MS m/z 174.0 (M+H).

### Preparation 3. Synthesis of 3-((tert-butyldiphenylsilyl)oxy)-2-fluoro-6-(methoxy-d₃)benzaldehyde

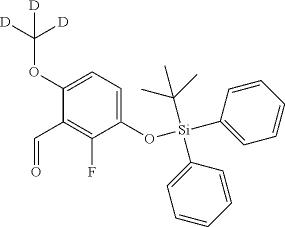 [SMILES: [2H]C([2H])([2H])Oc1ccc(O[Si](c2ccccc2)(c2ccccc2)C(C)(C)C)c(F)c1C=O]

Tert-butyl-[2-fluoro-4-(trideuteriomethoxy)phenoxy]-diphenyl-silane (11 g, 30 mmol) was dissolved in THF (130 mL) and cooled to −78° C. To the cooled solution, nBuLi (31 mL, 50 mmol, 1.6 M in hexanes) was added over 20 min. After 1.5 h, DMF (6.0 mL, 78 mmol) was added dropwise. The mixture was stirred at −78° C. for an additional 5 h. The reaction was quenched by the addition of satd aq NH₄Cl (25 mL). The mixture was allowed to warm to rt. After 18 h, the organic solvent was evaporated and the crude residue was extracted with 3× EtOAc. The combined organic extracts were washed with water and brine. The organic extracts were dried over Na₂SO₄, filtered and concentrated. The crude product was purified by normal phase purification, eluting with 0-40% EtOAc in hexanes to give the title compound (7.3 g, 60% yield). MS m/z 412.0 (M+H).

### Preparation 4. Synthesis of tert-butyl(2-fluoro-4-(methoxy-d₃)phenoxy)diphenylsilane

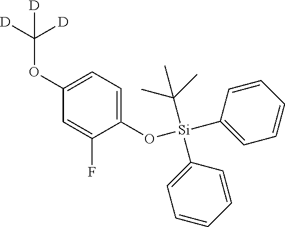 [SMILES: [2H]C([2H])([2H])Oc1ccc(O[Si](c2ccccc2)(c2ccccc2)C(C)(C)C)c(F)c1]

To a solution of 2-fluoro-4-(trideuteriomethoxy)phenol (9.2 g, 44 mmol) in DMF (100 mL) was added imidazole (4.5 g, 66 mmol), and tert-butylchlorodiphenylsilane (14 mL, 53 mmol). The reaction was stirred at rt for 2 d. The mixture was diluted with EtOAc, washed with 3× water and brine. The solution was dried over Na₂SO₄, filtered and evaporated to give the crude product. The crude product was purified by normal phase purification, eluting with 0-20% EtOAc in hexanes to give the title compound (15 g, 86% yield). MS m/z 400.9 (M+NH₄).

### Preparation 5. Synthesis of 2-fluoro-4-(methoxy-d₃)phenol

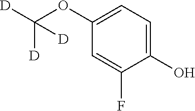 [SMILES: [2H]C([2H])([2H])Oc1ccc(O)c(F)c1]

To a pre-purged 70 mL Parr shaker bottle (N₂) was added 10% Pd/C (1.0 g, 9.7 mmol), followed by purgingagain with N₂. To the charged shaker, 250 mL MeOH was added, followed by 1-benzyloxy-2-fluoro-4-(trideuteriomethoxy)benzene (10 g, 44 mmol) in MeOH (250 mL). The bottle was sealed, purged with N₂, purged with H₂, and then pressurized to 60 psi H₂. The bottle was shaken at rt for 2 h, then depressurized and degassed with N₂. The suspension was filtered over celite with MeOH. The filtrate was concentrated to a crude residue. The crude product was purified by normal phase purification, eluting with 0-20% EtOAc in hexanes to give the title compound (9.2 g, 93% yield). 1H NMR (399.80 MHz, DMSO). 9.22 (s, 1H), 6.86 (dd, J=8.9, 10.1 Hz, 1H), 6.78 (dd, J=3.0, 13.0 Hz, 1H), 6.58 (ddd, J=8.9, 3.0, 1.4 Hz, 1H).

### Preparation 6. Synthesis of 1-(benzyloxy)-2-fluoro-4-(methoxy-d₃)benzene

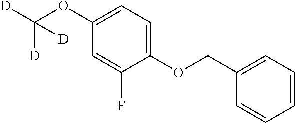 [SMILES: [2H]C([2H])([2H])Oc1ccc(OCc2ccccc2)c(F)c1]

A suspension of 4-benzyloxy-3-fluoro-phenol (10 g, 46 mmol), Cs₂CO₃ (22 g, 69 mmol) and trideuterio(iodo)methane (3.1 mL, 50 mmol) in DMF (100 mL) was stirred at rt for 18 h. The reaction was diluted with EtOAc, washed with 3× water and brine. The solution was dried over Na₂SO₄, filtered and evaporated to give the crude product. The crude product was purified by normal phase purification, eluting with 0-20% EtOAc in hexanes to give the title compound (10 g, 96% yield). MS m/z 253.0 (M+NH₄).

### Preparation 7. Synthesis of tert-butyl (3-((3-(1,3-dioxolan-2-yl)-4-ethyl-2-fluorophenoxy)methyl)phenyl)carbamate

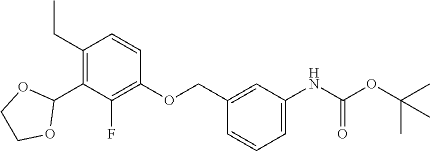 [SMILES: CCc1ccc(OCc2cccc(NC(=O)OC(C)(C)C)c2)c(F)c1C1OCCO1]

To a pre-purged 70 mL Parr shaker bottle (N₂) was added 5% Pd/C (32 mg, 0.16 mmol), followed by purging again with N₂. To the charged shaker, 2.5 mL EtOAc was added and then tert-butyl N-[3-[[3-(1,3-dioxolan-2-yl)-2-fluoro-4-vinyl-phenoxy]methyl]phenyl]carbamate (110 mg, 0.25 mmol) in EtOAc (2.5 mL). The bottle was sealed, purged with N₂, purged with H₂, and then pressurized to 60 psi H₂. The bottle was shaken at rt for 8 h, then depressurized and degassed with N₂. The suspension was filtered over celite with EtOAc. The filtrate was concentrated to give the title compound (89 mg, 84% yield). MS m/z 434.8 (M+NH₄)

### Preparation 8. Synthesis of tert-butyl (3-((3-(1,3-dioxolan-2-yl)-2-fluoro-4-vinylphenoxy)methyl)phenyl)carbamate

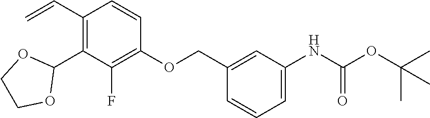 [SMILES: C=Cc1ccc(OCc2cccc(NC(=O)OC(C)(C)C)c2)c(F)c1C1OCCO1]

Tert-butyl N-[3-[[4-bromo-3-(1,3-dioxolan-2-yl)-2-fluoro-phenoxy]methyl]phenyl]carbamate (400 mg, 0.85 mmol), potassium vinyltrifluoroborate (0.14 g, 1.0 mmol) and Cs₂CO₃ (0.84 g, 2.6 mmol) were placed in a 25 mL microwave tube. The tube was purged with N₂ and THF (9 mL) and water (1 mL) were added. The solution was degassed by bubbling sub-surface N₂ for 5 min. palladium(II) acetate (10 mg, 0.042 mmol) was added, the tube capped, and mixture was heated at 100° C. for 18 h. Upon cooling to rt, EtOAc and H₂O were added. The phases were separated and the aqueous layer was extracted with 2×EtOAc. The combined organics were dried over Na₂SO₄, filtered and concentrated to a crude residue. The crude product was purified by normal phase purification, eluting with 50-100% DCM in hexanes to give the title compound (110 mg, 29% yield). MS m/z 432.8 (M+NH₄).

### Preparation 9. Synthesis of tert-butyl (3-((4-bromo-3-(1,3-dioxolan-2-yl)-2-fluorophenoxy)methyl)phenyl)carbamate

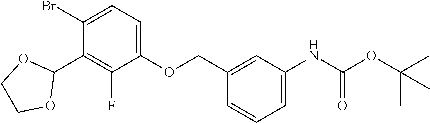 [SMILES: CC(C)(C)OC(=O)Nc1cccc(COc2ccc(Br)c(C3OCCO3)c2F)c1]

With a Dean-Stark trap attached, a solution of tert-butyl N-[3-[(4-bromo-2-fluoro-3-formyl-phenoxy)methyl]phenyl]carbamate (1.0 g, 2.4 mmol), ethylene glycol (0.55 mL, 9.8 mmol) and p-toluenesulfonic acid monohydrate (47 mg, 0.25 mmol) in toluene (16 mL) was refluxed at 135° C. After 1 h, the reaction solution was cooled to rt, washed with H₂O (15 mL) and EtOAc (25 mL). The phases were separated, and the aqueous layer was extracted 1× with EtOAc. The combined organic layers were dried over Na₂SO₄, filtered and concentrated to a crude residue. The crude product was purified by normal phase purification, eluting with 04% MeOH in DCM to give the title compound (500 mg, 44% yield). MS m/z 467.4 (M−H).

### Preparation 10. Synthesis of tert-butyl (3-((4-bromo-2-fluoro-3-formylphenoxy)methyl)phenyl)carbamate

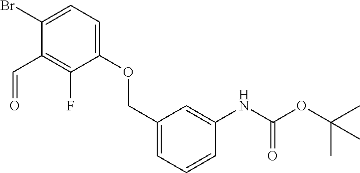 [SMILES: CC(C)(C)OC(=O)Nc1cccc(COc2ccc(Br)c(C=O)c2F)c1]

To a solution of 6-bromo-2-fluoro-3-hydroxy-benzaldehyde (5 g, 23 mmol) and Cs₂CO₃ (15 g, 46 mmol) in DMF (76 mL) was added tert-butyl N-[3-(bromomethyl)phenyl]carbamate (7.2 g, 25 mmol). After 18 h at rt, the mixture was diluted with EtOAc and water. The organic layer was washed with 3× water, brine, dried over Na₂SO₄, filtered and concentrated to a crude residue. The crude product was purified by normal phase purification, eluting with 0-20% EtOAc in hexanes to give the title compound (8.2 g, 85% yield). MS m/z 441.2 (M+NH₄).

### Preparation 11. Synthesis of tert-butyl (4-(2-fluoro-3-formyl-4-methoxyphenethyl)phenyl)carbamate

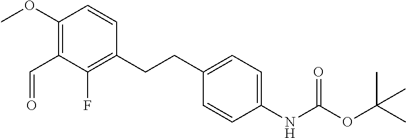 [SMILES: COc1ccc(CCc2ccc(NC(=O)OC(C)(C)C)cc2)c(F)c1C=O]

Tert-butyl N-[4-[2-[2-fluoro-3-(hydroxymethyl)-4-methoxy-phenyl]ethyl]phenyl]carbamate (2.1 g, 5.7 mmol) in DCM (60 mL) was cooled to 0° C. and treated with Dess-Martin periodinane (3.0 g, 7.1 mmol). After 1 h, the reaction was quenched with satd aq NaHCO₃ (4 mL) and Na₂S₂O₃ (0.5 M in water, 4 mL). The organic solvent was removed and the solution was diluted with EtOAc and water. The organic layer was washed with satd aq NaHCO₃, water, brine, and dried over MgSO₄, filtered, and concentrated to a crude residue. The crude product was purified by normal phase purification, eluting with 10-100% DCM in hexanes to give the title compound (1.2 g, 58% yield). MS m/z 391.0 (M+NH₄).

### Preparation 12. Synthesis of tert-butyl (4-(2-fluoro-3-(hydroxymethyl)-4-methoxyphenethyl)phenyl)carbamate

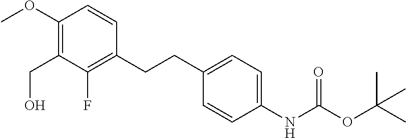 [SMILES: COc1ccc(CCc2ccc(NC(=O)OC(C)(C)C)cc2)c(F)c1CO]

To a cooled solution (0° C.) of methyl 3-[2-[4-(tert-butoxycarbonylamino)phenyl]ethyl]-2-fluoro-6-methoxy-benzoate (2.5 g, 6.2 mmol) in THF (31 mL) was added DIBAL (22 mL, 22 mmol, 1 mol/L in heptane) and let warm to rt. After 1.5 h, the reaction was cooled back to 0° C. and treated with 6 mL of satd aq NaHCO₃. The ice bath was removed, and the mixture vigorously stirred; the resulting gel was diluted with 3 ml of water and EtOAc and vigorously stirred to achieve a biphasic mixture. The slurry was diluted with EtOAc and the mixture filtered through a pad of celite with additional EtOAc. The filtrate was placed in a separatory funnel and washed with water and brine. The combined organics were dried over MgSO₄, filtered, and concentrated to give the title compound (2.1 g, 79% yield). MS m/z 373.8 (M−H).

### Preparation 13. Synthesis of methyl 3-(4-((tert-butoxycarbonyl)amino)phenethyl)-2-fluoro-6-methoxy benzoate

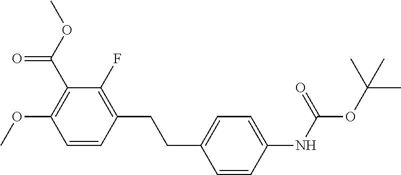 [SMILES: COC(=O)c1c(OC)ccc(CCc2ccc(NC(=O)OC(C)(C)C)cc2)c1F]

To methyl 3-[2-(4-aminophenyl)ethyl]-2-fluoro-6-methoxy-benzoate (2.5 g, 7.0 mmol) in DCM (10 mL) was added tBuOH (24 mL), DIPEA (1.5 mL, 8.7 mmol) and di-tert-butyl dicarbonate (1.8 g, 8.4 mmol). The reaction was heated to 50° C. After 1 h, the reaction was cooled to rt and concentrated to a crude residue. The material was dissolved in EtOAc and washed with 2×0.5N HCl, satd aq NaHCO₃, and brine. The combined organic layers were dried over MgSO₄, filtered, and concentrated. The crude product was purified by normal phase purification, eluting with 0-10% EtOAc in DCM to give the title compound (2.5 g, 89% yield). MS m/z 421.2 (M+NH₄).

### Preparation 14. Synthesis of methyl 3-(4-aminophenethyl)-2-fluoro-6-methoxybenzoate

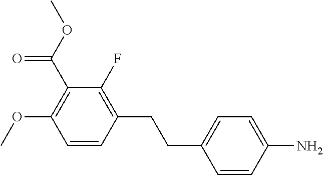 [SMILES: COC(=O)c1c(OC)ccc(CCc2ccc(N)cc2)c1F]

To a pre-purged 500 mL Parr shaker bottle (N₂) was added 10% Pd/C (380 mg, 0.36 mmol), followed by purging again with N₂. To the charged shaker, 65 mL MeOH was added and then methyl 2-fluoro-6-methoxy-3-[(E)-2-(4-nitrophenyl)vinyl]benzoate (2.5 g, 7.5 mmol) in MeOH (60 mL). The bottle was sealed, purged with N₂, purged with H₂, and then pressurized to 60 psi H₂. The bottle was shaken at rt for 3 h, then depressurized and degassed with N₂. The suspension was filtered over celite with MeOH. The filtrate was concentrated to give the title compound (2.5 g, 86% yield). MS m/z 303.8 (M+H).

### Preparation 15. Synthesis of methyl (E)-2-fluoro-6-methoxy-3-4-nitrostyryl)benzoate

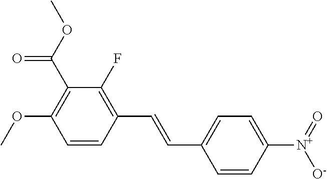 [SMILES: COC(=O)c1c(OC)ccc(/C=C/c2ccc([N+](=O)[O-])cc2)c1F]

A solution of methyl 2-fluoro-3-formyl-6-methoxy-benzoate (4.1 g, 20 mmol), diethyl 4-nitrobenzylphosponate (6.0 g, 22 mmol) and 2-methyl THF (200 mL) was cooled to −10° C., and was treated with potassium tert-butoxide (2.7 g, 23 mmol). After 1 h, the reaction was quenched with satd aq NH₄Cl and diluted with EtOAc. The organic layer was washed with water and brine. The combined organic layers were dried over MgSO₄, filtered, and concentrated. The crude product was purified by normal phase purification, eluting with 0-100% EtOAc in hexanes to give the title compound (5.6 g, 83% yield). MS m/z 332.0 (M+H).

### Preparation 16. Synthesis of tert-butyl (4-(2-(2-fluoro-3-formyl-4-methoxyphenoxy)ethyl)phenyl)carbamate

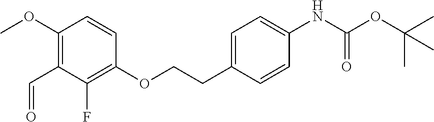 [SMILES: COc1ccc(OCCc2ccc(NC(=O)OC(C)(C)C)cc2)c(F)c1C=O]

To 2-fluoro-3-hydroxy-6-methoxy-benzaldehyde (500 mg, 2.9 mmol) and 2-[4-(tert-butoxycarbonylamino)phenyl]ethyl 4-methylbenzenesulfonate (1.4 g, 3.5 mmol) in MeCN (18 mL) was added K₂CO₃ (1.2 g, 8.8 mmol) and the suspension was stirred at 80° C. for 16 h. The reaction mixture was diluted with EtOAc and washed with water and brine. The combined organic layers were dried over MgSO₄, filtered, and concentrated. The crude product was purified normal phase purification, eluting with 0-30% EtOAc in hexanes to give the title compound (0.9 g, 70% yield). MS m/z 387.8 (M−H).

### Preparation 17. Synthesis of 4-((tert-butoxycarbonyl)amino)phenethyl 4-methylbenzenesulfonate

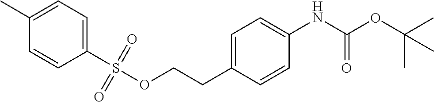 [SMILES: Cc1ccc(S(=O)(=O)OCCc2ccc(NC(=O)OC(C)(C)C)cc2)cc1]

To a solution of tert-butyl N-[4-(2-hydroxyethyl)phenyl]carbamate (2.5 g, 11 mmol) in DCM (50 mL) was added Et₃N (2.9 mL, 21 mmol), followed by p-toluenesulfonyl chloride (2.3 g, 12 mmol) and DMAP (130 mg, 1.0 mmol). The reaction mixture was stirred at rt for 18 h. The reaction mixture was diluted with DCM and washed with water and brine. The combined organic layers were dried over Na₂SO₄, filtered and concentrated under reduced pressure to give a crude residue. The crude product was purified normal phase purification, eluting with 0-25% EtOAc in hexanes to give the title compound (3.9 g, 94% yield). MS m/z 408.9 (M+NH₄).

### Preparation 18. Synthesis of tert-butyl (3-(2-(2-fluoro-3-formyl-4-methoxyphenoxy)ethyl)phenyl)carbamate

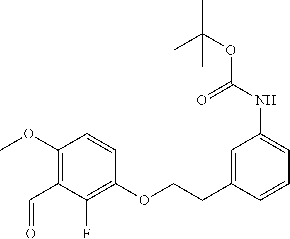 [SMILES: COc1ccc(OCCc2cccc(NC(=O)OC(C)(C)C)c2)c(F)c1C=O]

To 2-fluoro-3-hydroxy-6-methoxy-benzaldehyde (600 mg, 3.5 mmol) and 2-[3-(tert-butoxycarbonylamino)phenyl]ethyl 4-methylbenzenesulfonate (1.5 g, 3.7 mmol) in MeCN (20 mL) was added K₂CO₃ (1.5 g, 11 mmol) and the resulting suspension was stirred at 80° C. for 20 h. The reaction mixture was diluted with EtOAc and washed with water and brine. The combined organic layers were dried over MgSO₄, filtered and concentrated under reduced pressure to give a crude residue. The crude product was purified normal phase purification, eluting with 0-25% EtOAc in hexanes to give the title compound (0.96 g, 62% yield). MS m/z 388.4 (M−H).

### Preparation 19. Synthesis of 3-tert-butoxycarbonyl)amino)phenethyl 4-methylbenzenesulfonate

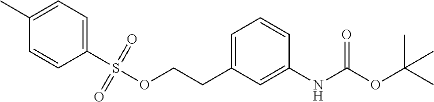 [SMILES: Cc1ccc(S(=O)(=O)OCCc2cccc(NC(=O)OC(C)(C)C)c2)cc1]

To a solution of tert-butyl N-[3-(2-hydroxyethyl)phenyl]carbamate (2.7 g, 11 mmol) in DCM (60 mL) was Et₃N (3.2 mL, 23 mmol) followed by p-toluenesulfonyl chloride (2.5 g, 13 mmol) and DMAP (140 mg, 1.1 mmol). The reaction mixture was stirred at rt for 18 h. The reaction was diluted with DCM and washed with satd aq NH₄Cl and brine. The combined organic layers were dried over MgSO₄, filtered and concentrated under reduced pressure to give a crude residue. The crude product was purified normal phase purification, eluting with 0-25% EtOAc in hexanes to give the title compound (3.0 g, 67% yield). MS m/z 389.8 (M−H).

### Preparation 20. Synthesis of tert-butyl (3-(2-hydroxyethyl)phenyl)carbamate

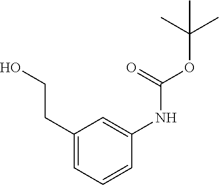 [SMILES: CC(C)(C)OC(=O)Nc1cccc(CCO)c1]

To a solution of 2-[3-(tert-butoxycarbonylamino)phenyl]acetic acid (3.0 g, 12 mmol) in THF (60 mL) was added dropwise borane-THF complex (17 mL, 17 mmol, 1 mol/L in THF) at 0° C., under N₂ atmosphere. The mixture was warmed to rt while stirring under N₂ for 16 h. The reaction was cooled to 0° C., MeOH was added, and the solution was warmed to rt. The crude reaction was concentrated under reduced pressure to give a crude residue. The crude residue was purified normal phase purification, eluting with 5-50% EtOAc in hexanes to give the title compound (2.7 g, 93% yield). MS m/z 254.8 (M+NH₄).

### Example 2. Synthesis of (1S,2S,4R,6S,8S,9S,11S,12S,13R)-6-[3-[(3-aminophenyl)methoxy]-2-fluoro-6-(trideuteriomethoxy)phenyl]-11-hydroxy-8-(2-hydroxyacetyl)-9,13-dimethyl-5,7-dioxapentacyclo[10.8.0.02,9.04,8.013,18]icosa-14,17-dien-16-one (Compound No. 1)

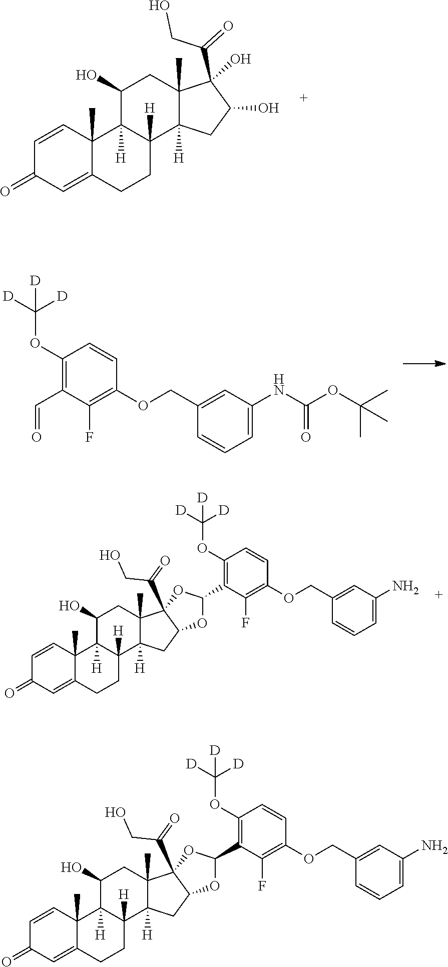

To a suspension of tert-butyl N-[3-[[2-fluoro-3-formyl-4-(trideuteriomethoxy)phenoxy]methyl]phenyl]carbamate (250 mg, 0.63 mmol) and (8S,9S,10R,11S,13S,14S,16R,17S)-11,16,17-trihydroxy-17-(2-hydroxyacetyl)-10,13-dimethyl-7,8,9,11,12,14,15,16-octahydro-6H-cyclopenta[a]phenanthren-3-one (250 mg, 0.66 mmol) in MeCN (6 mL) at −10° C. was added perchloric acid (70% in water, 0.29 mL, 5 equiv.) dropwise. The mixture was stirred at −10° C. for 1 h. The reaction was quenched cold, with the addition of sat'd aq NaHCO₃, and partitioned between water and 10% IPA/DCM. The phases were separated, and the aqueous layer was extracted 1× with 10% IPA/DCM. The combined organics were dried over MgSO₄, filtered and concentrated to give a crude solid. The solid was purified by reverse phase chromatography, eluting with 50-100% MeCN in 10 mM ammonium bicarbonate water+5% methanol to give the title compound (420 mg, 16 yield). MS m/z 637.4 (M+H).

¹HNMR (399.8 MHz, d₆-DMSO) δ 17.33-7.31 (i, 1H), 7.17-7.12 (nM, 1H), 6.99 (t, J=7.7 Hz, 1H), 6.73-6.70 (m, 1H), 6.58 (s, 1H), 6.53-6.46 (i, 3H), 6.19-6.16 (m, 1H), 5.95 (s, 1H), 5.25 (d, J=6.6 Hz, 1H), 5.10 (s, 2H), 5.00-4.93 (m, 3H), 4.80-4.78 (m, 1H), 4.37-4.30 (m, 2H), 4.04-3.98 (m, 1H), 2.37-2.31 (m, 1H), 2.11-2.07 (i, 2H), 1.89-1.82 (m, 4H), 1.62-1.59 (i, 1H), 1.40 (s, 3H), 1.26-1.24 (m, 2H), 0.87 (s, 3H).

### Example 3. Synthesis of Compound Nos. 2-5

Compound Nos. 2-5 were prepared essentially by the method of Example 2.

| | ES/MS | |
|---|---|---|
| | (m/z) | |
| Compound No. | (M + H) | ¹H NMR |
| 2 | 632.3 | 1H NMR (399.80 MHz, DMSO): 7.33 (d, J = 10.0 Hz, |
| | | 1H), 7.15-7.11 (m, 1H), 6.99 (t, J = 7.7 Hz, 1H), 6.90 |
| | | (d, J = 8.3 Hz, 1H), 6.58 (d, J = 1.7 Hz, 1H), 6.53-6.48 |
| | | (m, 2H), 6.30 (s, 1H), 6.20-6.16 (m, 1H), 5.95 (s, 1H), |
| | | 5.30 (d, J = 6.9 Hz, 1H), 5.10-5.07 (m, 3H), 5.00-4.93 |
| | | (m, 2H), 4.82-4.80 (m, 1H), 4.38-4.31 (m, 2H), 4.06- |
| | | 4.01 (m, 1H), 2.68-2.59 (m, 2H), 2.38-2.30 (m, 1H), |
| | | 2.11-2.02 (m, 2H), 1.90-1.79 (m, 4H), 1.70-1.65 (m, |
| | | 1H), 1.40 (s, 3H), 1.24-1.04 (m, 6H), 0.88 (s, 3H). |
| 3 | 632.2 | 1H NMR (400.13 MHz, DMSO): 7.32 (d, J = 10.0 Hz, |
| | | 1H), 7.18 (t, J = 8.6 Hz, 1H), 6.83 (d, J = 8.3 Hz, 2H), |
| | | 6.76-6.73 (m, 1H), 6.46 (d, J = 8.4 Hz, 3H), 6.19-6.16 |
| | | (m, 1H), 5.95 (s, 1H), 5.26 (d, J = 6.7 Hz, 1H), 4.96 (dd, |
| | | J = 5.5, 6.4 Hz, 1H), 4.82-4.77 (m, 3H), 4.40-4.30 (m, |
| | | 2H), 4.02-3.96 (m, 1H), 3.65 (s, 3H), 2.71-2.61 (m, |
| | | 3H), 2.34-2.31 (m, 1H), 2.08 (s, 4H), 1.88-1.75 (m, |
| | | 4H), 1.61-1.53 (m, 1H), 1.40 (s, 3H), 1.23-1.07 (m, |
| | | 3H), 0.88 (s, 3H). |
| 4 | 648.6 | 1H NMR (400.13 MHz, DMSO): 7.32 (d, J = 10.1 Hz, |
| | | 1H), 7.15-7.10 (m, 1H), 6.93 (d, J = 8.3 Hz, 2H), 6.73- |
| | | 6.71 (m, 1H), 6.50-6.45 (m, 3H), 6.18 (dd, J = 1.8, 10.1 |
| | | Hz, 1H), 5.95 (s, 1H), 5.76 (s, 1H), 5.24 (d, J = 6.6 Hz, |
| | | 1H), 4.97 (dd, J = 5.3, 6.5 Hz, 1H), 4.88 (s, 2H), 4.78 |
| | | (d, J = 3.2 Hz, 1H), 4.36-4.29 (m, 2H), 4.08-3.97 (m, |
| | | 3H), 3.63 (s, 3H), 2.81 (t, J = 7.2 Hz, 2H), 2.59-2.55 (m, |
| | | 1H), 2.34-2.31 (m, 1H), 2.11-2.02 (m, 2H), 1.88-1.75 |
| | | (m, 4H), 1.60-1.54 (m, 1H), 1.40 (s, 3H), 1.24-1.21 (m, |
| | | 2H), 0.87 (s, 3H). |
| 5 | 648.6 | 1H NMR (400.13 MHz, DMSO): 7.32 (d, J = 10.1 Hz, |
| | | 1H), 7.17-7.12 (m, 1H), 6.93 (t, J = 7.7 Hz, 1H), 6.74- |
| | | 6.71 (m, 1H), 6.47-6.40 (m, 4H), 6.18 (dd, J = 1.8, 10.1 |
| | | Hz, 1H), 5.95 (s, 1H), 5.76 (s, 1H), 5.24 (d, J = 6.6 Hz, |
| | | 1H), 5.00-4.96 (m, 3H), 4.78 (d, J = 3.3 Hz, 1H), 4.36- |
| | | 4.30 (m, 2H), 4.12-4.05 (m, 3H), 3.63 (s, 3H), 2.83 (t, |
| | | J = 7.1 Hz, 2H), 2.34-2.31 (m, 1H), 2.11-2.08 (m, 2H), |
| | | 1.88-1.76 (m, 4H), 1.61-1.56 (m, 1H), 1.40 (s, 3H), |
| | | 1.23-1.20 (m, 2H), 0.87 (s, 3H). |

### Example 4. hGR CoActivator Recruitment Assay

The activity of glucocorticoid compounds was measured using the LanthaScreen TR-Fret GR Coactivator Assay from Life Technologies (A15899). The compounds were acoustically transferred to an assay plate in a 3-fold 10-point serial dilution with atop concentration of 200 nM. Ten microliters of a 2× solution of GR-LBD was added to the compound plate and incubated for 10 min. Then ten microliters of a 2× solution of Fluoresein-SRC1-4 and Tb labelled anti-GST antibody were added to the plate. The plate was incubated in the dark for two hours and then read on an Envision plate reader, with excitation at 340 nm and emission at 520 nm (Fluorescein) and 490 nm (Terbium). The emission ratio of 520/490 was analyzed in Genedata. To obtain percent activity, the data was compared to a negative control of DMSO and positive control of 4 μM dexamethasone. The following exemplified compounds were tested following the procedure as essentially described above and exhibited the following activity as listed in Table 2.

TABLE 2
In vitro potency of compounds of Compound Nos.
1-5 in the hGR CoActivator Recruitment Assay

| Compound No. | EC₅₀ (nM) |
|---|---|
| 1 | 1.93 |
| 2 | 1.52 |
| 3 | 1.65 |
| 4 | 1.48 |
| 5 | 2.25 |

What is claimed is:

1. A compound of the formula: 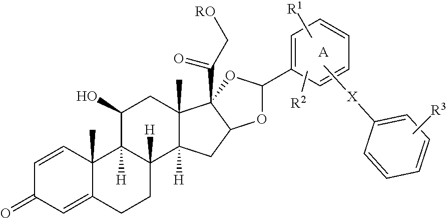
wherein R is H or 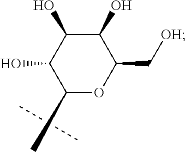
R¹ is H, halogen, CN, C1-C3 alkyl, C3-C6 cycloalkyl, C1-C3 alkoxy, C2-C3 alkenyl, OCF₃, 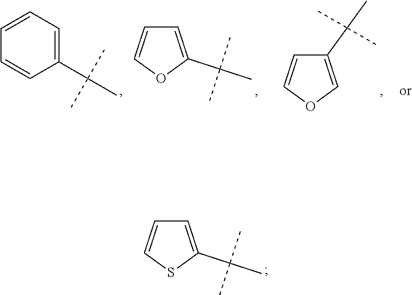
R² is H, halogen, C1-C3 alkyl, C1-C3 alkoxy, or C2-C3 alkenyl;
R³ is NH₂, or CH₂NH₂; and
X is O, OCH₂, OCH₂CH₂, CH₂O, SCH₂, CH₂S, CH₂, NHCH₂, CH₂NH, N(CH₃)CH₂, CH₂CH₂, C≡C, or a bond, wherein X is connected to phenyl ring A at the ortho or the meta position, or a pharmaceutically acceptable salt thereof.

2. The compound of claim 1, wherein R³ is NH₂, or a pharmaceutically acceptable salt thereof.

3. The compound of claim 1, wherein R is H, or a pharmaceutically acceptable salt thereof.

4. The compound of claim 1, wherein the compound is of Formula 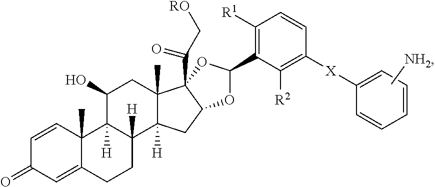
or a pharmaceutically acceptable salt thereof.

5. The compound of claim 1, wherein R¹ is F, CH₂CH₃, OCH₃, or OC(²H)₃, or a pharmaceutically acceptable salt thereof.

6. The compound of claim 1, wherein R¹ is F, or a pharmaceutically acceptable salt thereof.

7. The compound of claim 1, wherein R¹ is CH₂CH₃, or a pharmaceutically acceptable salt thereof.

8. The compound of claim 1, wherein R¹ is OCH₃, or a pharmaceutically acceptable salt thereof.

9. The compound of claim 1, wherein R¹ is OC(²H)₃, or a pharmaceutically acceptable salt thereof.

10. The compound of claim 1, wherein R² is F, CH₂CH₃, OCH₃, or OC(²H)₃, or a pharmaceutically acceptable salt thereof.

11. The compound of claim 1, wherein R² is F, or a pharmaceutically acceptable salt thereof.

12. The compound of claim 1, wherein R² is CH₂CH₃, or a pharmaceutically acceptable salt thereof.

13. The compound of claim 1, wherein R² is OCH₃, or a pharmaceutically acceptable salt thereof.

14. The compound of claim 1, wherein R² is OC(²H)₃, or a pharmaceutically acceptable salt thereof.

15. The compound of claim 1, wherein X is CH₂CH₂, OCH₂, or OCH₂CH₂, or a pharmaceutically acceptable salt thereof.

16. The compound of claim 1, wherein X is CH₂CH₂, or a pharmaceutically acceptable salt thereof.

17. The compound of claim 1, wherein X is OCH₂, or a pharmaceutically acceptable salt thereof.

18. The compound of claim 1, wherein X is OCH₂CH₂, or a pharmaceutically acceptable salt thereof.

19. The compound of claim 1, wherein the compound is: 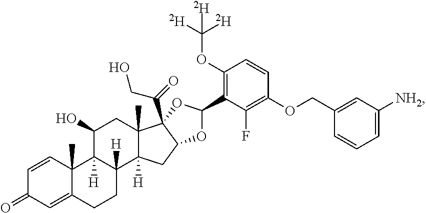
or a pharmaceutically acceptable salt thereof.

20. The compound of claim 19, wherein the compound is:

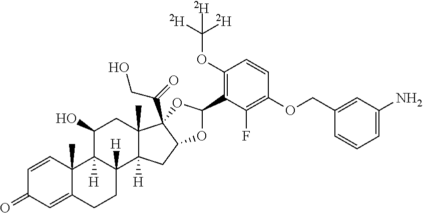

21. The compound of claim 1, wherein the compound is: 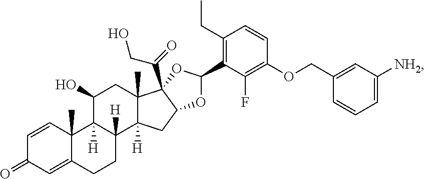
or a pharmaceutically acceptable salt thereof.

22. The compound of claim 21, wherein the compound is:

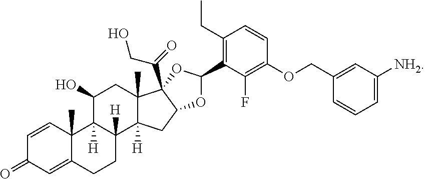

23. The compound of claim 1, wherein the compound is: 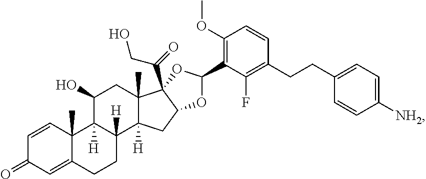
or a pharmaceutically acceptable salt thereof.

24. The compound of claim 23, wherein the compound is:

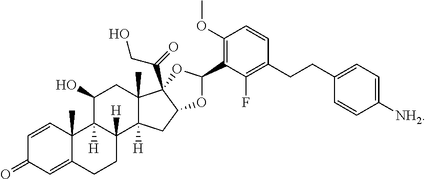

25. The compound of claim 1, wherein the compound is: 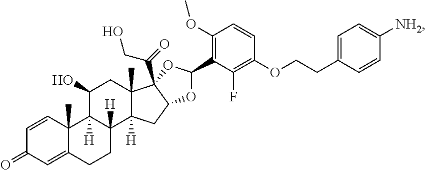
or a pharmaceutically acceptable salt thereof.

26. The compound of claim 25, wherein the compound is: 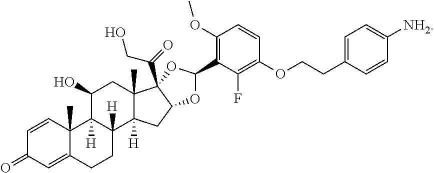
or a pharmaceutically acceptable salt thereof.

27. The compound of claim 1, wherein the compound is: 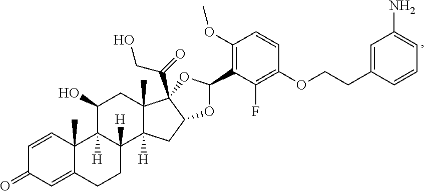
or a pharmaceutically acceptable salt thereof.

28. The compound of claim 27, wherein the compound is:

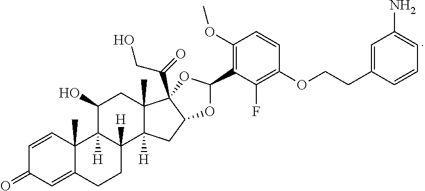

29. The compound of claim 1, wherein the compound is: 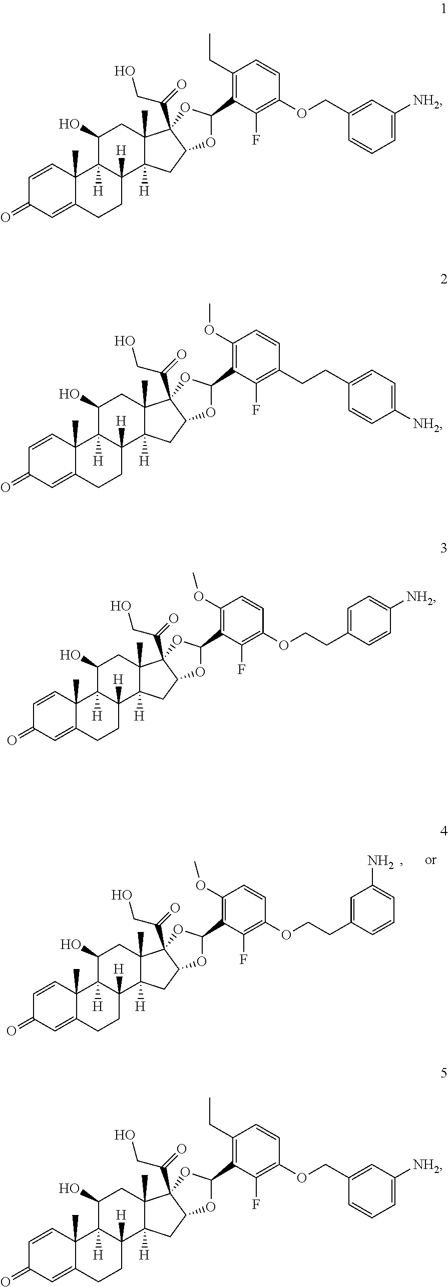
or a pharmaceutically acceptable salt thereof.

30. A method of treating atopic dermatitis in a patient, comprising administering to a patient in need of such treatment an effective amount of the compound of claim 1, or a pharmaceutically acceptable salt thereof.

31. A method of treating rheumatoid arthritis in a patient, comprising administering to a patient in need of such treatment an effective amount of the compound of claim 1, or a pharmaceutically acceptable salt thereof.

32.-36. (canceled)

37. A pharmaceutical composition, comprising the compound, or pharmaceutically acceptable salt thereof, of claim 1, and one or more pharmaceutically acceptable carrier, diluent, or excipient.

38. A process for preparing a pharmaceutical composition, comprising admixing a compound, or pharmaceutically acceptable salt thereof, of claim 1 with one or more pharmaceutically acceptable carrier, diluent, or excipient.
